(19) United States
(12) Patent Application Publication
(10) Pub. No.: US 2006/0021621 A1
(43) Pub. Date: Feb. 2, 2006

(54) Sterile environments

(76) Inventor: Hans Rudolph Kriek, Bussum (NL)

Correspondence Address: DIEDERIKS & WHITELAW, PLC, 12471 DILLINGHAM SQUARE, #301, WOODBRIDGE, VA, 22192, US

(21) Appl. No.: 10/527,691

(22) PCT Filed: Sep. 11, 2003
(86) PCT No.: PCT/GB03/03925

§ 371 Date: Mar. 11, 2005

(30) Foreign Application Priority Data
Sep. 11, 2002 (GB) 0221069.8

(51) Int. Cl. A61B 19/00; A61B 19/08

(52) U.S. Cl. 128/849; 128/851; 128/852

(57) ABSTRACT

An apparatus includes two or more sterile environments, each of which is at least partially enclosed by a wall member. A portion of the wall member of at least one of the environments is joined to a portion of the wall member of another one of the environments and the wall members at or within the joined portions are capable of being opened to connect the sterile environments. At least a portion of one or more of the surfaces that form the joined portions is provided with a bactericidal substance to sterilize the joined portions before they are opened. The wall members are preferably joined by an adhesive in which the bactericidal substance may be impregnated. The sterile environments may be an operating enclosure, a window unit or a carrier containing surgical instruments, fluids, medicines, a glove, blood or saline.

This invention relates to a method of connecting sterile environments, to apparatus and enclosures for use in the method and, in particular, to an enclosed operating environment to which external sterile enclosures or the like may be attached.

The maintenance of sterile environments during surgery on the human or animal body is of primary importance in preventing infections arising during surgery and improving the success rate of surgical operations. Generally, surgical theatres are kept as clean as possible to prevent infections during surgery and, both for use in surgical theatres and outside of such environments, various apparatus and systems have been suggested for maintaining a sterile environment in which surgical procedures can be carried out.

In particular, it has been proposed to perform operations in sterile enclosures, such as tents or clean rooms, the inside of which have previously been sterilised and can be kept clean. Such sterile enclosures have been proposed not only to allow performance of surgical operations outside of surgical theatres but also for use in surgical theatres and the like to reduce the time spent in sterilising the theatres and also generally to reduce the likelihood of infections arising.

However, no previously suggested sterile enclosure is capable of remaining entirely free from contamination. In particular, sterile enclosures can be contaminated on the introduction of items, such as surgical instruments or medication, for example, from outside the enclosure during surgical procedures. This generally occurs because there is no entirely reliable system of conveniently entering and exiting the enclosure without contaminating the sterile environment within.

According to the present invention there is therefore provided a method of connecting two or more sterile environments each of which are at least partially enclosed by a wall member, comprising joining a portion of the wall member of one environment to a portion of the wall member of another environment and opening the wall members at or within the joined portions to connect the sterile environments, wherein at least one surface of the wall members that meet to make the join is provided with a bactericidal substance to sterilise the join before opening.

Also according to the present invention there is provided apparatus comprising two or more sterile environments, each of which are at least partially enclosed by a wall member, a portion of the wall member of at least one of the environments having means for joining it to a portion of the wall member of another environment and the wall members at or within the joined portions being capable of being opened to connect the sterile environments, wherein at least one surface of the wall members that meet to make the join is provided with a bactericidal substance to sterilise the join before opening.

Thus, a surgical procedure can be carried out in one of the sterile environments and, for example, surgical instruments or the like contained in another sterile environment can be introduced into the sterile environment in which the surgical procedure is being performed without contaminating that sterile environment. For example, a package containing the surgical instruments might be joined to the enclosure in which the surgical procedure is being performed, the surfaces of the package and enclosure which are joined being sterilised using a bactericidal substance, and the joined portions of the wall members of the enclosure and package opened, for example by cutting, to connect the enclosure and package and, in this example, to allow access to the surgical instruments from inside the enclosure.

There is reduced possibility of contamination of the sterile environments (e.g. enclosure and package) firstly because the sterile environments are only connected by opening the join between the environments and neither environment is therefore open to the external environment at any time, and secondly because the outer surfaces of wall members of the sterile environments which form the join can be sterilised with the bactericidal substance to sterilise the join before opening such that any portion of the join which has been exposed to the external environment is sterilised before opening.

The sterile environments may be joined and connected in various ways. For example, joining means comprising press fit fastenings, Velcro® or the like may be provided in desired positions on the sterile environments to allow the wall members to be joined. Similarly, preformed openings such as zips, and snap-fit closures may be provided in the area of the joining means to allow opening of the join to connect the environments.

However, in a particularly preferred embodiment, the joining means comprises an adhesive. The adhesive may be provided on a wall member of the sterile environment as an adhesive tape or layer. Preferably, the adhesive has a backing layer which may be peeled off when it is required to join the sterile environments.

The joining means preferably further comprises a perforable membrane. For example, a section of the wall member of one or more of the sterile environments may comprise a perforable membrane with an adhesive layer around its periphery. Thus, after the sterile environments have been joined by the adhesive, the membrane(s) of the or each environment may be perforated to connect the environments. A scalpel or the like may be used to facilitate the perforation.

However, it is possible that using an easily perforable membrane may weaken the wall member of the sterile environment. Thus, in a particularly preferred embodiment, the means for joining the sterile environments comprises a membrane having a cleft sealed by a weaker membrane. The weaker membrane may then be broken to connect the sterile environments after the join has been made. This has the advantage of ensuring the opening is made in a desired place and the opening being easy to make without compromising the strength of the wall member of the sterile environment.

Also, in a preferred embodiment, the joining means may further comprise means for resealing the opening after the sterile environments have been connected and means for detaching the sterile environments after the opening has been resealed. For example, a closure, e.g a snap fit closure, may be provided behind the joining means of one of the sterile environments which can be closed to reseal the opening. The wall member of one sterile environment may then be severed outwardly of the closure to remove that sterile environment whilst the other sterile environment remains intact and isolated from the outside environment.

The substance provided on the surfaces that meet to make the join can comprise any bactericidal, bacteriological or sterilising agent and can be applied in a variety of ways. For example, a bactericidal gas may be contained in a capsule which is perforated as the side walls are joined such that the side walls that meet to make the join are sterilised. However, in a particularly preferred example, the substance provided on the surfaces that meet to make the join comprises a bactericidal substance, such as iodine, impregnated into the joining means, e.g. impregnated into the wall members, membrane or adhesive layer. Thus, for example, as the adhesive sticks to make the join, the surfaces which meet to make the join are sterilised.

The applicants have appreciated from the above that the joining means and substance provided on the surfaces to be joined can be carried on both the sterile environments or on only one or other sterile environment to be connected. Where the joining means and substance provided on the surfaces to be joined are carried on only one or other of the sterile environments, the wall member of sterile environment not carrying the joining means or substance may comprise a perforable membrane. In a particularly preferred embodiment, the sterile environment comprises an enclosure formed by a perforable membrane. Thus, in either case, the sterile environment carrying the joining means and substance may be connected to the other sterile environment at any point on the wall member or, indeed, whole enclosure. This provides a particularly flexible system which is very convenient to use as the sterile environments can be connected to allow optimum access between the connected environments.

According to a third aspect of the present invention there is therefore provided a peripheral sterile environment comprising a wall member having means for joining it to a flexible membrane of a main sterile environment, wherein at least one surface of the wall member and/or membrane that meet to make the join is provided with a bactericidal substance to sterilise the join before severing the membrane within the join to connect the sterile environments.

As mentioned above this provides a particularly flexible system as the peripheral sterile environment may be joined to the main sterile environment as desired, e.g. at any position on the membrane of the main sterile environment which may be enclosed entirely by the membrane. Thus, for example a package containing surgical instruments may be joined to the enclosure in which the surgical procedure is being performed at the most convenient location for the surgeon.

Although each sterile environment might contain any item which is required to be kept sterile or can have performed in it any operation, surgical or otherwise, that requires absolute cleanliness or isolation, in a particularly preferred embodiment, one of the sterile environments is an operating enclosure in which surgery may be carried out.

The operating enclosure may take any form, such as a rigid or collapsible sealed container. However, it is preferred that the enclosure comprises a collapsible bubble, which may be of a plastics material. A collapsible bubble is particularly straightforward to manufacture such that its inside conveniently be kept sterile and, as it may be supplied in a collapsed state, it is easily transportable. In preparation for use, the bubble may be filled with air or gas to inflate it such that it is suitable for the performance of a surgical procedure.

The enclosure or bubble when inflated may be sufficiently rigid to support itself. Alternatively, it may be required to inflate the bubble such that the air or gas within the bubble is at a pressure above atmospheric pressure and supports the bubble, or to support the bubble in some other way. In a particularly preferred embodiment a frame is provided to support the enclosure. The frame may be external to the enclosure. This allows the enclosure or bubble to be attached to or to be contained by the frame to keep it in place during performance of the surgical procedure.

The frame may be attached to a surgical trolley or bed on which the patient lies and, in a particularly preferred embodiment, the frame comprises one or more arches slidably mounted over a surgical trolley.

The enclosure is usually filled with sterile air or gas, whether for inflation of the enclosure or not. Additionally or alternatively, the gas or air may have a bactericidal substance added to it in order to ensure maintenance of sterility. As another way to help to maintain sterility, the gas or air may be continually recirculated through a sterilising filter. A pump and filter arrangement may therefore be attached to the operating enclosure for this purpose.

The operating enclosure may be optically clear or transparent such that a surgeon can observe the inside of the operating enclosure to perform a surgical procedure. However, this may not always be the case and, where it is not easy for the surgeon to see into the operating enclosure, the apparatus may preferably further comprise one or more windows which can be provided on the operating enclosure to assist viewing the inside of the enclosure.

In a particularly preferred embodiment, one of the sterile environments is a window unit comprising a window with a cover on one side. The window under the cover is sterile such that the cover can be joined to the wall member of another sterile environment and the cover and wall member opened to such that the other sterile environment can be viewed directly through the window without obstruction by the cover or wall member.

The window may comprise an optically clear sheet, for example made of plastics material or the like. The means for joining the window unit to a portion of the wall member of the operating enclosure may comprise an adhesive layer applied to the cover, the cover may be a perforable membrane and/or the bactericidal substance provided on the surfaces that meet to make the join may be impregnated into the adhesive.

According to a fourth aspect of the present invention there is therefore provided a window unit comprising a window on one side of which a cover is provided having means for joining it to the flexible membrane of a sterile environment, wherein at least one surface of the cover and/or membrane that meet to make the join is provided with a bactericidal substance to sterilise the join before severing the membrane within the join to connect the window to the sterile environment.

The cover may therefore be joined to the side wall of the operating enclosure and, when the join is severed, the cover and the side wall of the operating enclosure joined to the cover may be parted or removed such that the interior of the operating enclosure can be viewed directly through the window rather than the side wall of the enclosure. In doing this, the sterile environment of the operating enclosure is not exposed to any external contamination as at no time is the enclosure open to the external environment, and the surfaces of the cover and side wall that have been exposed to the external environment and are opened to connect the window to the enclosure are sterilised before opening.

In order to perform any surgical procedure it is, of course, necessary to have access to the patient. Conventionally, a patient is covered with surgical drapes during an operation and a hole is left in the drapes around the area of the intended incision through which the surgical procedure is to be carried out. This area is cleaned and has a bactericidal substance applied to it to reduce the risk of infection after the incision has been made. However, infection can still occur due to contamination after the bactericidal substance has been applied or from the surrounding environment directly into the incision after it has been made.

The Applicant has recognised that the chance of infection can be reduced by one of the sterile environments comprising a drape that at least partially encloses the area of the intended incision. Thus, the area may be prepared and a drape applied immediately that seals the area and keeps it sterile. The operating enclosure may be connected to the drape at a later stage, when the patient is ready for surgery, to form an operating environment with access to the area of the intended incision without risk of infection between preparation and the connection of the operating enclosure.

The drape may comprise a sheet having an opening for surrounding the area of the intended incision and a cover over the opening for sealing the opening. The joining means may comprise an adhesive layer on the cover, the cover may be a perforable membrane and the substances provided on the surfaces that meet to make the join to sterilise the join before opening may comprise a bactericidal substance impregnated into the adhesive.

The area of the intended incision may therefore be prepared and treated with a bactericidal substance, the drape attached to the patient with the opening over the area of the intended incision, the enclosure later joined to the drape and the drape connected to the enclosure such that a surgeon can gain access to the area of the intended incision from inside the enclosure. Thus, the area of the intended incision is not exposed to anything but a sterile environment after preparation.

In order to perform a surgical procedure, the surgeon must also, of course, have access to the operating enclosure. In the prior art, gloves have been built into wall of operating enclosures to receive a surgeons hands. However, these often provide only restricted access to the enclosure as they are only provided at certain points on the operating enclosure and can therefore make surgery cumbersome and difficult by making it difficult to reach all areas of the enclosure easily.

In a preferred embodiment of the present invention, one of the sterile environments may therefore comprise a glove carrier. For example, the exterior of a glove may be enclosed by an enclosure and joining means may be provided at the end of the enclosure proximal to the fingers of the glove. Thus, the enclosure of the glove carrier can be connected to the operating enclosure and the surgeon can insert his hand into the glove and extend the glove into the enclosure, such that he can manipulate items within the operating enclosure. The glove may therefore be connected to the operating enclosure where desired such that a surgeon can arrange easy access to the desired area(s) of the operating enclosure.

Thus, according to a fifth aspect of the present invention there is provided a glove carrier comprising a glove, the exterior of which is enclosed by an enclosure and the interior of which is freely accessible, the enclosure having means for joining it to the flexible membrane of a sterile environment, wherein at least one surface of the enclosure and/or flexible membrane that meet to make the join is provided with a bactericidal substance to sterilise the join before severing the membrane within the join to connect the exterior of the glove to the sterile environment.

Other sterile environments may be comprise carriers containing, for example, surgical instruments, fluids or medicines for administering during the surgical procedure. These may be connected to the operating enclosure such that the contents can be accessed from inside the enclosure, whilst the entire environment remains sterile.

Thus, according to a sixth aspect of the present invention there is provided an isolated operating environment comprising: an operating enclosure; a drape for communicating the operating enclosure with a patient; and one or more peripheral enclosures for connecting to the operating enclosure, the drape and one or more peripheral enclosures each having a wall member or membrane with means for joining them to respective portions of a wall member of the operating enclosure, the wall member or membrane at the joined portions being capable of being opened to communicate the peripheral enclosures and patient with the operating enclosure, wherein at least one of the surfaces that meet to make each of the joins is provided with a bactericidal substance to sterilise the joins before opening.

A further aspect of the invention provides a method of providing a sterile operating environment, comprising placing a drape over part of a patient, providing an enclosure over the drape, adhering the drape and a wall member of the enclosure together by means of bactericidal treated adhesive or tape, adhering a wall member of the operating enclosure to at least one peripheral enclosure by means of bactericidal treated adhesive or tape, and providing intercommunication openings in regions of the drape and wall member which are confined by or beneath the adhesive or adhesive tape.

An operating environment is therefore provided in which the area of the patient in which it is intended to make an incision only comes into contact with one or more interconnected sterile environments from the time of preparation and attaching of the drape to the end of the operation. The used sterile environments remaining at the end of the operation may be disposed of, if desired, with the result that nobody is exposed to contamination from the patient. The possibility of infections arising from surgical procedures is therefore vastly reduced, as well as the safety of those performing operations increased.

A further aspect of the invention provides a surgical drape having an initially sealed opening and provided with bactericidal treated adhesive or adhesive tape adjacent the opening for connecting the drape with a further sterile environment in use.

A further aspect of the invention provides a flexible membrane for confining a sterile environment, the membrane being provided with a bactericidal adhesive or bactericidal adhesive tape which extends over or around a region of the membrane which defines, or can be ruptured to define, an opening in the membrane in use.

Preferred embodiments of the present invention will now be described, by way of example only, with reference to the accompanying drawings, in which:

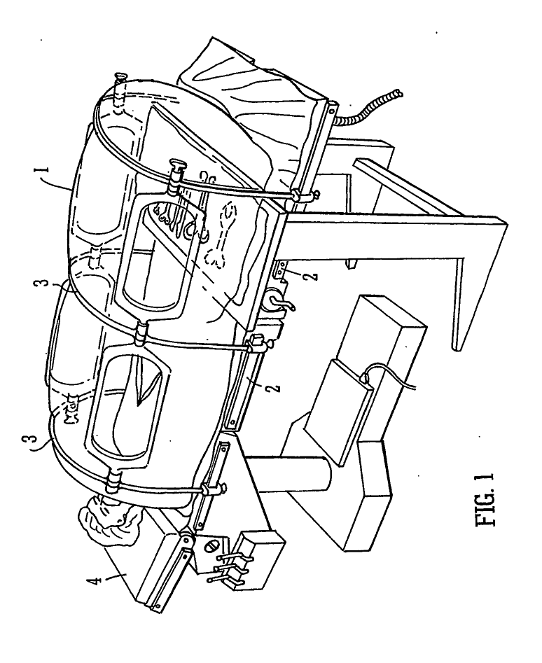

FIG. 1 is an illustration of an operating environment in accordance with the invention;

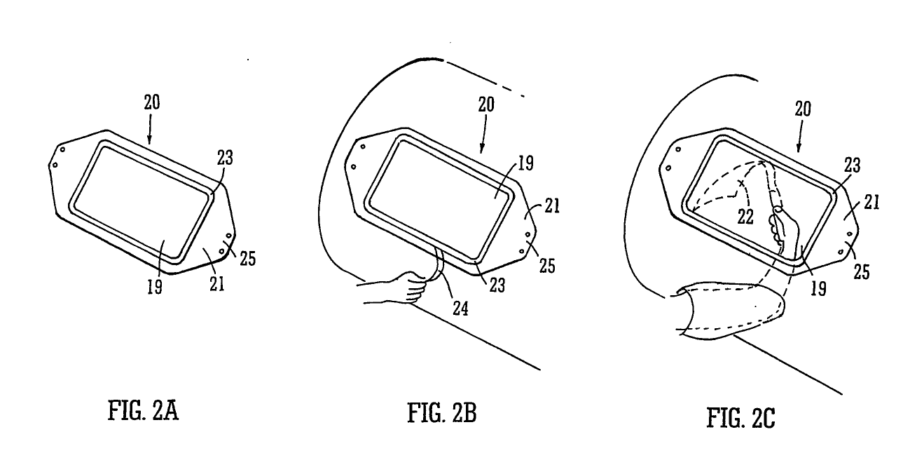

FIGS. 2A to 2C are illustrations of a window for attaching to the operating environment of the invention;

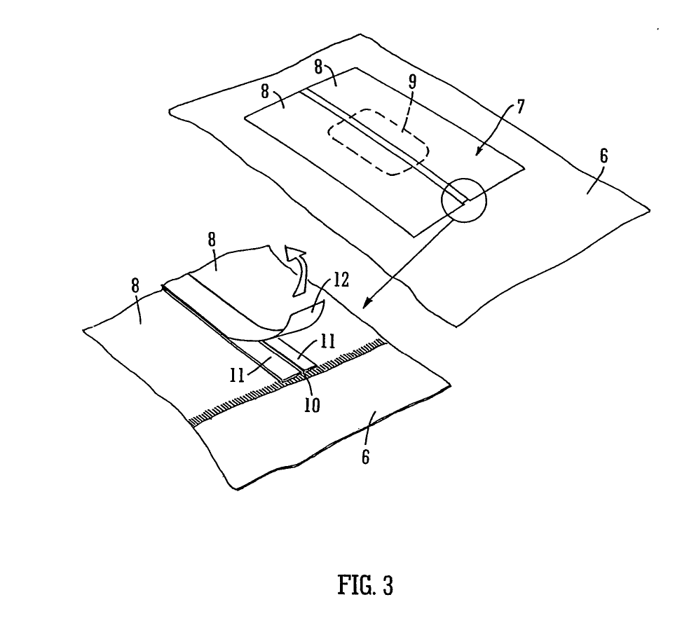

FIG. 3 is an illustration of a general drape in accordance with the present invention;

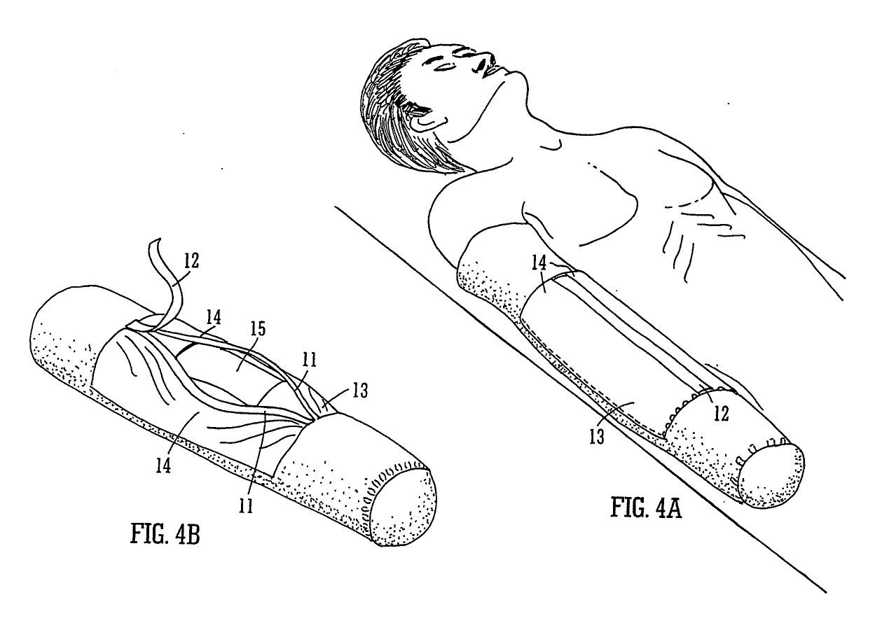

FIGS. 4A and 4B are illustrations of a limb drape in accordance with the invention;

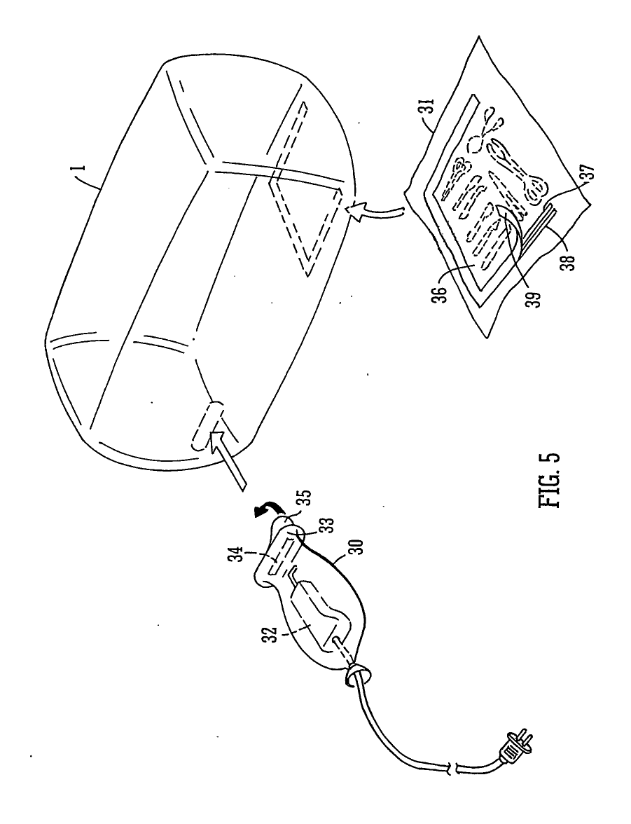

FIG. 5 is an illustration of the operating environment of the invention along with some attachments; and

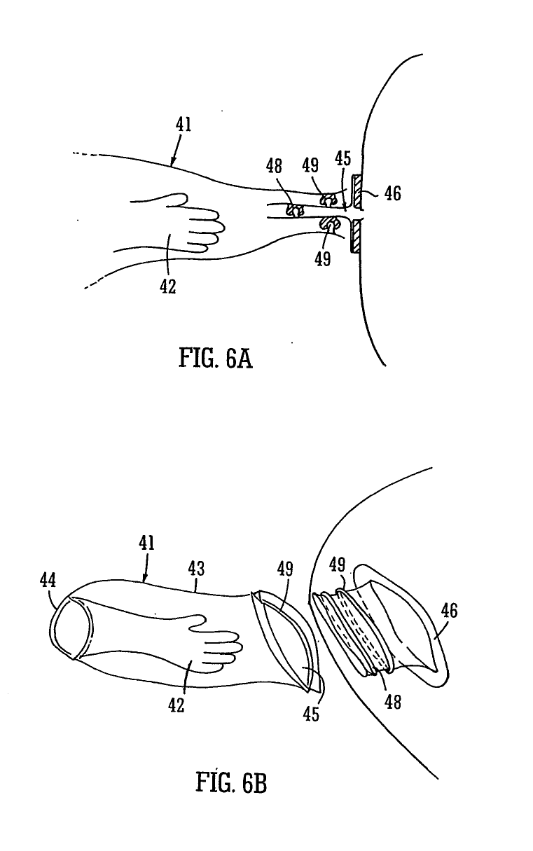

FIGS. 6A and 6B are illustrations of a glove attachment in accordance with the present invention.

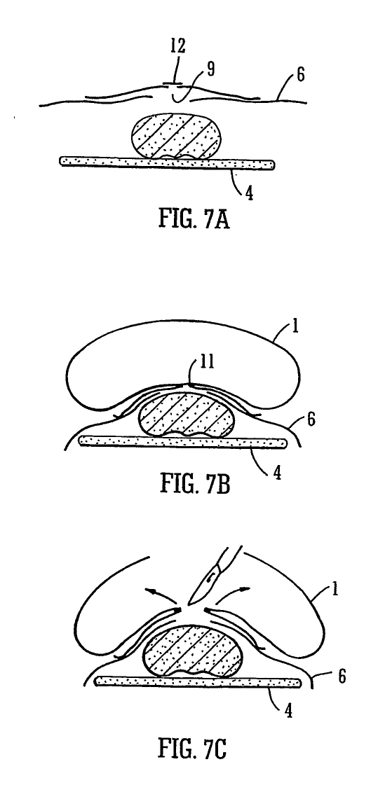

FIGS. 7A to 7C are illustrations of the general drape of FIG. 3 at various steps during placement of the drape; and

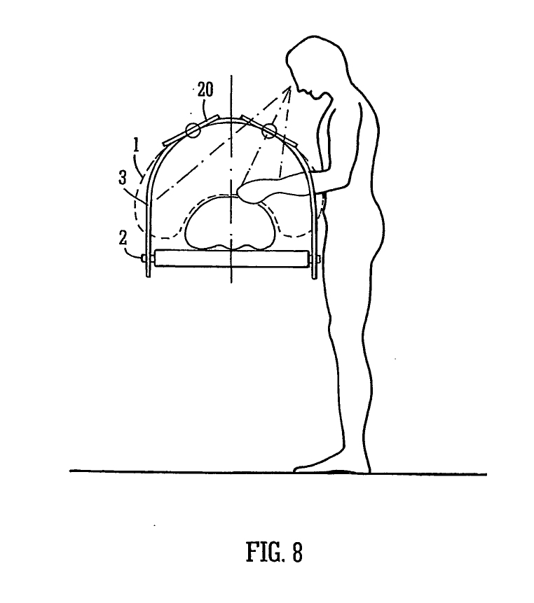

FIG. 8 is an illustration of the operating environment of the invention with the window of FIGS. 2A to 2C in position;

Referring to FIG. 1, an operating environment comprises an enclosure 1 which, in the example shown, is supported by a frame.

The frame comprises tracks 2 and supporting arches 3. The tracks 2 run along either side of the length of an operating trolley or bed 4 and the supporting arches 3 are mounted on and extend between the tracks 2 to form a tunnel over the operating trolley 4. In this example, the arches 3 are slidably mounted on the tracks 2 in order that they can be moved to different positions along the length of the trolley 4 to suitably support the enclosure 1.

The enclosure 1 fits in the tunnel, i.e. the space under the arches 3 and on top of the operating trolley 4, and is supported by the arches 3. In this example, when the enclosure 1 is in use, air or gas is supplied to the inside of the enclosure 1 at a pressure, sufficiently greater than atmospheric pressure to inflate the enclosure 1 such that it fills the tunnel or space and is thus merely supported by the arches 3 to the extent that they hold it in place in the tunnel. In other examples, the enclosure 1 may be sufficiently rigid to support its own weight that the gas or air is not required to be pressurised. In a yet further example, the enclosure 1 may be supported by being attached to the arches 3 and the air or gas supplied at any required pressure.

In this example, inflation of the enclosure 1 is maintained by a pump (not shown) which contains a filter for sterilising the air which fills the enclosure 1.

The pump is arranged such that sterilised air is continually circulated during the operation to further improve the maintenance of sterility within the enclosure 1.

Referring to FIGS. 2A to 2C, one or more windows 20 can be attached to the enclosure by the surgeon to give him a clearer view of the part of the patients body upon which he wishes to operate. In this example, each window 20 comprises a perspex or transparent plastics sheet 19 with a support 21. The side of the window 20 which is to be attached to the enclosure 1 has a membrane 22 on its surface and an adhesive tape 23 into which a bactericidal substance is impregnated. The adhesive tape 23 has a backing 24 which may be peeled off to expose the tape 23. In this example, the membrane 22 is impregnated with a bactericidal substance. In another example, the membrane 22 is also adhesive and covered by the backing 24.

The support 21 of the window 20 is provided with tabs 25 to which clips (not shown) may be attached. The clips may, in turn, be attached to the arches 3 of the frame to position the window 20, as shown in FIG. 3, such that the surgeon may easily view the area to be operated on.

Referring to FIG. 3, a general drape 6 covers a patient during various surgical operations, particularly those performed on the abdomen or torso. The drape 6 has an access panel 7 comprising two covers 8 covering an opening 9 in the material of the drape 6 below the covers 8. The covers 8 meet over the opening 9 where they are attached by a membrane 10. An adhesive tape 11, into which a bactericidal substance is impregnated, is provided on the covers 8 at each side of the membrane 10. The adhesive tape 11 is covered with a backing 12 which may be peeled off to expose the tape 11.

Referring to FIGS. 4A and 4B, a limb drape 13 covers a patient during surgical operations on a limb, such as a right arm for example, as shown in FIG. 4A. The limb drape 13 is sized to fit a limb of the patient rather than the torso. Similarly to the general drape 6, the limb drape 13 comprises two covers 14 which cover an opening 15 in material below the covers 14. The covers 14 meet over the opening 15 where they are attached by a membrane (not shown). Adhesive tape 11, into which a bacteriological substance is impregnated, is provided on the covers 14 at each side of the membrane 16. The adhesive tape 11 is covered with a backing 12 which may be peeled off to expose the tape Only the part of the patient's body on which the operation is to be performed is covered by a drape 6,13. The remainder of the patient's body is not required to be draped, but remains isolated from the operating environment by the enclosure 1, after attachment to the drape as described below.

Referring to FIG. 5, two attachments 30, 31 or peripheral enclosures are shown which are able to be attached to the enclosure 1. The first attachment 30 carries a container 32, which contains a fluid such as blood or saline for administering to the patient, in a sterile environment. The attachment 30 has an adhesive surface 33 at one end, impregnated with a bactericidal substance and having an opening 34 sealed with a membrane (not shown). The adhesive surface 33 and opening 34 are covered with a removable backing 35.

The second attachment 31 carries a variety of surgical implements in a sterile environment. A cover 36 is provided over the surgical instruments which has an opening 37 around its periphery. The opening 37 is sealed with a membrane (not shown) and surrounded by an adhesive tape 38, impregnated with a bactericidal substance. A removable backing 39 is provided over the adhesive tape.

Referring to FIGS. 6A and 6B a glove attachment 41 or carrier houses a glove 42. An enclosure 43 extends from the cuff 44 of the glove 42 around the outside of the glove 42 to form the carrier 41. Thus, a surgeon is free to put his hand into the glove 42, but the outer surface of the glove 42 is sealed.

The end of the glove attachment 41 distal from the cuff 44 or proximal to the fingers of the glove 42 has an opening 45 surrounded by an adhesive tape 46, impregnated with a bactericidal substance. The opening 45 is sealed with a membrane (not shown) and the adhesive tape 46 has a removable backing tape (not shown) over it. In addition, this end of the glove attachment 41 has means for removing the glove from the enclosure 1. A resealable seal 48 is provided in the attachment 41, inwardly of the opening 45 that can seal the opening 45 once the membrane has been broken. The glove attachment 41 is also removable outwardly of this resealable seal 48 by releasing seals 49 provided outward of the resealable seal 48.

In use, the area around the incision intended to be made to perform the operation is prepared by treating it with a bactericidal substance. As quickly as possible, a drape 6,13 is then placed over the area such that the opening 9,15 is over the area of the intended incision. The area is thus isolated and should remain sterile. This is shown schematically in FIG. 7A.

The enclosure 1 is prepared by inflating it as necessary and inserting it under or attaching it to the arches 3 of the frame as desired. In this example, the enclosure 1 is then attached to the drape 6,13 by removing the backing tape 12 to expose the adhesive tape 11 and sticking the outside of enclosure 1 to the adhesive tape 11. This is shown schematically in FIG. 7B.

One or more, and usually at least two, glove attachments 41 are then attached to the outside of the enclosure 1 by removing the backing tape (not shown) and exposing the adhesive tape 46 on the end of the glove attachment(s) 41. The glove attachment(s) 41 can then be attached where desired on the outside of the enclosure 1. The surgeon is thus able to select the location(s) that will enable the easiest access to the area where the surgical operation is to be performed.

Access may then be gained by the surgeon to the interior of the operating enclosure 1 by inserting his hand in the glove 42, piercing the membrane sealing the opening 45 and the wall of the enclosure 1 and extending the glove into the enclosure 1. Where the membrane is difficult to pierce or split by hand, an implement, such as a small scalpel, is provided in the glove enclosure 43 for use to pierce the membrane 47.

Once the surgeon has gained access to the enclosure 1, referring to FIG. 7C, the enclosure 1 may be perforated above, the membrane 10 of the drape 6,13 along with the membrane 10 itself. Thus, the enclosure 1 is connected to the area of the intended incision such that the surgical operation may be performed.

The window 20 is attached to the arches 3 in a position allowing the surgeon to be conveniently able to see through the window 20, as shown in FIG. 8. The adhesive tape 23 is then removed from the window 20 so that the window is stuck to the outside surface of the enclosure 1. The portion of the enclosure 1 under the window 20 is then cut away by the surgeon, thus removing both the membrane 22 and a section of the side wall of the enclosure 1 under the enclosure such that the surgeon can see through the window. The adhesive tape 23 ensures an airtight seal around the opening in the side of the enclosure 1. Thus, the surgeon may observe the part of the patients body upon which he is working inside the enclosure directly through the window 20, which is optionally clearer than the membrane of the enclosure 1.

Any of the other attachments 30, 31 which are desired to be used are also attached to the enclosure 1. The first attachment 30 is attached by removing the backing 35 to expose the adhesive surface 33 and sticking the adhesive surface to the outside of the enclosure 1 when desired. The bactericidal substance impregnated into the adhesive surface sterilises the outside surface of the membrane of the enclosure 1 where the adhesive surface 33 is stuck. The surgeon perforates the membrane of the enclosure 1 adjacent the opening 34 of the attachment 30, along with the membrane sealing the opening 34. Thus, access is gained from inside the enclosure 1 to the container 32 inside the attachment 30 without risk of contaminating the enclosure 1.

Similarly, the second attachment 31 can be attached to the enclosure 1 by removing the backing 39 and sticking the adhesive tape 38 to the outside surface of the membrane of the enclosure 1. Again, the surgeon perforates the membrane of the enclosure 1 adjacent the opening 37 of the attachment 31, along with the membrane sealing the opening 37 to gain access to the surgical instruments under the cover 36.

1. An apparatus comprising two or more sterile environments, each of which are at least partially enclosed by a wall member, a portion of said wall member of at least one of said environments having means for joining it to a portion of said wall member of another environment, and said wall members at or within the joined portions being capable of being opened to connect the sterile environments, wherein at least a portion of one or more of the surfaces that form said joined portions is provided with a bactericidal substance which sterilizes the join before opening.

2. An apparatus as claimed in claim 1, wherein said joining means comprises an adhesive.

3. An apparatus as claimed in claim 1, wherein said joining means is in the form of an adhesive tape or layer provided on the wall member of one or both of said sterile environment.

4. An apparatus as claimed in claim 3, wherein said adhesive tape or layer comprises a backing layer which may be peeled off.

5. An apparatus as claimed in claim 1, wherein the wall members are openable at or within the joined portion by perforating a membrane.

6. An apparatus as claimed in claim 5, wherein the means for joining the sterile environments comprises a membrane having a rupturable cleft.

7. An apparatus as claimed in claim 1, wherein said bactericidal substance is a bactericidal gas contained in a capsule.

8. An apparatus as claimed in claim 1, wherein said bactericidal substance is impregnated into said joining means.

9. An apparatus as claimed in claim 1, wherein at least one of said sterile environments comprises an enclosure formed by a perforable membrane.

10. An apparatus as claimed in claim 1, wherein at least one of said sterile environments comprises a carrier containing, surgical instruments, fluids, medicines, a glove, blood or saline.

11. An apparatus as claimed in claim 1, wherein one of said sterile environments is an operating enclosure in which surgery may be carried out.

12. An apparatus as claimed in claim 1, wherein at least one of said sterile environments comprises a collapsible bubble made of a plastics material.

13. An apparatus as claimed in claim 12, wherein means are provided to fill the bubble with air or gas to inflate it such that it is suitable for the performance of a surgical procedure.

14. An apparatus as claimed in claim 1, wherein a frame is provided to support at least one of said sterile environments.

15. An apparatus as claimed in claim 1, wherein said frame comprises one or more arches slidably mounted over a surgical trolley.

16. An apparatus as claimed in claim 1, wherein means are provided for filling at least one of said sterile environments with sterile air or gas.

17. An apparatus as claimed in claim 16, wherein said air or gas has a bactericidal substance added to it.

18. An apparatus as claimed in claim 1, wherein one of said sterile environments is a window unit comprising a window with a cover on one side.

19. An apparatus as claimed in claim 18, wherein said means for joining said window unit to a portion of a wall member of the other sterile environment comprises an adhesive layer applied to said cover, said adhesive layer being impregnated with a bactericidal substance.

20. An apparatus as claimed in claim 1, wherein said joining means further comprises means for resealing an opening after the sterile environments have been connected and means for detaching the sterile environments after said opening has been resealed.

21. A peripheral sterile environment comprising a wall member having means for joining it to a flexible membrane of a main sterile environment, wherein at least a portion of a surface on or adjacent the wall member is provided with a bactericidal substance which sterilizes the region of join before severing or opening said membrane within said join to communicate said sterile environments with another.

22. A window unit comprising a window on one side of which a cover is provided having means for joining it to a flexible membrane of a sterile environment, wherein at least a portion of one or more of the surfaces which form the join is provided with a bactericidal substance to sterilize said join before severing said membrane within said join to connect said window to said sterile environment.

23. A glove carrier comprising a glove, the exterior of said glove enclosed by an enclosure and the interior of said glove accessible to a user, said enclosure having means for joining it to a flexible membrane of a sterile environment, wherein at least a portion of one or more of surfaces in the region of the join is provided with a bactericidal substance which sterilize the region of the join in use before severing or opening said membrane within said join to connect said exterior of said glove to said sterile environment.

24. A flexible membrane for confining a sterile environment, there being provided a bactericidal substance such as an adhesive or bactericidal adhesive tape which extends over or around a region of said membrane which region is adapted to be opened or ruptured to define an opening in said membrane in use.

25. An isolation operating environment comprising, an operating enclosure, a drape for communicating the operating enclosure with a patient, and one or more peripheral enclosures for connecting to the operating enclosure, said drape and one or more peripheral enclosures each having a wall member or membrane with means for joining them to respective portions of a wall member of said operating enclosure, said wall member or membrane at the joined portions being capable of being opened to communicate said peripheral enclosures and patient with said operating enclosure, wherein at least a portion of one or more surfaces which meet to form the joins is provided with a bactericidal substance to sterilize said joins before opening.

26. A surgical drape having an initially sealed opening and provided with bactericidal treated adhesive or adhesive tape adjacent said opening for joining said drape with a further sterile environment in use.

27. A surgical drape as claimed in claim 26, wherein said opening is sealed by a cover and said adhesive or adhesive tape is provided on said cover.

28. A surgical drape as claimed in claim 27, wherein said cover is a perforable membrane.

29. A method of connecting two or more sterile environments each of which are at least partially enclosed by a wall member, comprising joining a portion of said wall member of one environment to a portion of said wall member of another environment and opening said wall members at or within the joined portions to connect said sterile environments, wherein at least a portion of one or more of the surfaces that form the joined portions is provided with a bactericidal substance to sterilize the region of join before opening.

30. A method of providing a sterile operating environment, comprising placing a drape over part of a patient, providing an enclosure over said drape, adhering said drape and a wall member of said enclosure together by means of bactericidal treated adhesive or tape, adhering said wall member of said enclosure to at least one peripheral enclosure by means of bactericidal treated adhesive or tape, and providing intercommunication openings in regions of said drape and wall member which are confined by or beneath said adhesive or adhesive tape.
